(19) United States
(12) Patent Application Publication
(10) Pub. No.: US 2023/0041261 A1
(43) Pub. Date: Feb. 9, 2023

(54) Introducer Components, Assemblies, and Methods Thereof

(71) Applicant: Bard Access Systems, Inc., Salt Lake City, UT (US)

(72) Inventor: Glade H. Howell, Draper, UT (US)

(21) Appl. No.: 17/883,490

(22) Filed: Aug. 8, 2022

Related U.S. Application Data
(60) Provisional application No. 63/231,092, filed on Aug. 9, 2021.

(51) Int. Cl. A61M 39/02; A61M 25/09; A61M 5/32

(52) U.S. Cl. CPC A61M 39/0247; A61M 25/09; A61M 5/3287; A61M 5/3293; A61M 2039/0258; A61M 2039/0264; A61M 2039/0626

(57) ABSTRACT

Disclosed are introducer components, assemblies, and methods. For example, an introducer assembly can include a fluidly connected syringe and needle. The needle can include a needle shaft and a needle hub over a proximal portion of the needle shaft. The needle hub can include a port and a valve disposed in the port. The port can be in a side of the needle hub proximal of a proximal end of the needle shaft. The valve can be configured to form a fluid-tight seal around an elongate medical device such as an access guidewire when passed through the port and into a needle-hub lumen of the needle hub. Such an access guidewire can be disposed in the introducer assembly just proximal of a needle tip of the needle. In this way, the access guidewire can be immediately advanced into a blood-vessel lumen upon establishing a needle tract thereto with the needle.

### PRIORITY

This application claims the benefit of priority to U.S. Provisional Patent Application No. 63/231,092, filed Aug. 9, 2021, which is incorporated by reference in its entirety into this application.

### BACKGROUND

A guidewire is typically placed in a blood vessel with an introducer assembly before inserting a central venous catheter (“CVC”) or the like into the blood vessel over the guidewire. The introducer assembly typically includes a needle connected to a syringe. Upon accessing the blood vessel with the needle, the needle must be disconnected from the syringe to allow insertion of the guidewire into the needle through a needle hub thereof and, subsequently, into the blood vessel. Disconnecting the needle from the syringe as well as inserting the guidewire into the needle risk puncturing a backwall of the blood vessel, losing access to the blood vessel, or both due to overhandling the needle. What is needed is an introducer assembly that does not require disconnecting the needle from the syringe for inserting the guidewire into the blood vessel.

Disclosed herein are introducer components, assemblies, and methods that address the foregoing.

### SUMMARY

Disclosed herein is an introducer assembly including, in some embodiments, a syringe and a needle fluidly connected to the syringe. The needle includes a needle shaft and a needle hub over a proximal portion of the needle shaft. The needle hub includes a port and a valve disposed in the port. The port is in a side of the needle hub proximal of a proximal end of the needle shaft. The valve is configured to form a fluid-tight seal around an elongate medical device when the medical device is passed through the port and into a needle-hub lumen of the needle hub.

In some embodiments, the introducer assembly further includes an access guidewire slidably disposed in the introducer assembly in a ready-to-deploy state of the introducer assembly. The access guidewire passes through the port, through the needle-hub lumen, through the proximal end of the needle shaft, and into a needle-shaft lumen of the needle shaft such that a guidewire tip in a distal portion of the access guidewire is disposed just proximal of a needle tip in a distal portion of the needle shaft.

In some embodiments, the guidewire tip is a ‘J’-shaped guidewire tip. The guidewire tip assumes a straightened state in the ready-to-deploy state of the introducer assembly and a curved state when the guidewire tip is advanced beyond the needle tip in a deployed state of the introducer assembly.

In some embodiments, the access guidewire includes a bare-wire portion and a wound-wire portion proximal of the bare-wire portion. The bare-wire portion distally extends through the valve in at least the ready-to-deploy state of the introducer assembly for forming the fluid-tight seal.

In some embodiments, the access guidewire includes a proximal portion proximally extending from the port in the ready-to-deploy state of the introducer assembly. The proximal portion of the access guidewire is disposed in a sterile barrier configured to maintain sterility of the access guidewire.

In some embodiments, the valve includes a split septum compressed in the port.

In some embodiments, the needle hub further includes a needle-hub connector. The needle-hub connector includes a needle-hub bore proximal of the port. A syringe tip of the syringe is disposed in the needle-hub bore, thereby fluidly connecting the needle to the syringe.

Also disclosed herein is an introducer needle including, in some embodiments, a needle shaft and a needle hub over a proximal portion of the needle shaft. The needle hub includes a port and a valve disposed in the port. The port is in a side of the needle hub proximal of a proximal end of the needle shaft. The valve is configured to form a fluid-tight seal around an elongate medical device when the medical device is passed through the port and into a needle-hub lumen of the needle hub.

In some embodiments, the valve includes a split septum compressed in the port.

In some embodiments, the needle hub further includes a needle-hub connector. The needle-hub connector includes a needle-hub bore proximal of the port. The needle-hub bore is configured to accept a syringe tip inserted therein for fluidly connecting the needle to the syringe.

Also disclosed herein is a method for securing vascular access. The method includes, in some embodiments, an introducer assembly-obtaining step, a needle tract-establishing step, and an access guidewire-advancing step. The introducer assembly-obtaining step includes obtaining an introducer assembly. The introducer assembly includes a syringe, a needle fluidly coupled to the syringe, and an access guidewire slidably disposed in the introducer assembly. The needle includes a needle hub having a port in a side of the needle hub. The port is proximal of a proximal end of a needle shaft disposed in the needle hub. The access guidewire passes through a valve disposed in the port, through a needle-hub lumen of the needle hub, through the proximal end of the needle shaft, and into a needle-shaft lumen of the needle shaft. The needle tract-establishing step includes establishing a needle tract from an area of skin to a blood-vessel lumen of a patient with the needle. The access guidewire-advancing step includes advancing the access guidewire into the blood-vessel lumen for the securing of the vascular access.

In some embodiments, the method further includes an introducer assembly-adjusting step. The introducer assembly-adjusting step includes adjusting the introducer assembly such that the introducer assembly is in a ready-to-deploy state thereof. In the ready-to-deploy state of the introducer assembly, a guidewire tip in a distal portion of the access guidewire is disposed just proximal of a needle tip in a distal portion of the needle shaft for performing the access guidewire-advancing step immediately upon the establishing of the needle tract in the needle tract-establishing step.

In some embodiments, the access guidewire-advancing step allows the guidewire tip of the access guidewire to transition from a straightened state in the access-guidewire passageway to a curved state in the blood-vessel lumen.

In some embodiments, the method further includes a plunger-withdrawing step. The plunger-withdrawing step includes withdrawing a plunger from a barrel of the syringe to create a slight vacuum before reaching the blood-vessel lumen. The slight vacuum ensures blood flashes back into at least a syringe tip to confirm the establishing of the needle tract in the needle tract-establishing step.

In some embodiments, the method further includes a blood-aspirating step. The blood-aspirating step includes aspirating blood with the syringe to confirm the establishing of the needle tract in the needle tract-establishing step. The valve disposed in the port is configured to form a fluid-tight seal around a bare-wire portion of the access guidewire for maintaining a vacuum during the blood-aspirating step.

In some embodiments, the method further includes a needle-withdrawing step. The needle-withdrawing step includes withdrawing the needle from the patient leaving the access guidewire in the blood-vessel lumen.

In some embodiments, the needle-withdrawing step includes holding the guidewire in place at or near the area of skin including the needle tract while withdrawing the needle over a proximal portion of the access guidewire.

In some embodiments, the method further includes a syringe-and-needle disconnecting step. The syringe-and-needle disconnecting step includes disconnecting the needle from the syringe before the needle-withdrawing step.

In some embodiments, the method further includes an air-bleeding step. The air-bleeding step includes bleeding air into the port during the needle-withdrawing step by pushing the access guidewire to a side of the port against the valve. The air-bleeding step of the bleeding of air into the port obviates the disconnecting step of disconnecting the needle from the syringe.

In some embodiments, the valve includes a split septum compressed in the port.

These and other features of the concepts provided herein will become more apparent to those of skill in the art in view of the accompanying drawings and following description, which describe particular embodiments of such concepts in greater detail.

### DRAWINGS

FIG. 1 illustrates a perspective view of an introducer assembly in accordance with some embodiments.

FIG. 2 illustrates a side view of the introducer assembly in accordance with some embodiments.

FIG. 3 illustrates a top view of the introducer assembly in accordance with some embodiments.

FIG. 4 illustrates an exploded view of the introducer assembly in accordance with some embodiments.

FIG. 5 illustrates a top view of an introducer needle in accordance with some embodiments.

FIG. 6 illustrates a side view of the introducer needle in accordance with some embodiments.

FIG. 7 illustrates a longitudinal cross section of the introducer needle in accordance with some embodiments.

FIG. 8 illustrates a longitudinal cross section of the introducer needle including an access guidewire disposed therein in accordance with some embodiments.

FIG. 9 illustrates a portion of a method of using the introducer assembly in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, a guidewire is typically placed in a blood vessel with an introducer assembly before inserting a CVC or the like into the blood vessel over the guidewire. The introducer assembly typically includes a needle connected to a syringe. Upon accessing the blood vessel with the needle, the needle must be disconnected from the syringe to allow insertion of the guidewire into the needle through a needle hub thereof and, subsequently, into the blood vessel. Disconnecting the needle from the syringe as well as inserting the guidewire into the needle risk puncturing a backwall of the blood vessel, losing access to the blood vessel, or both due to overhandling the needle. What is needed is an introducer assembly that does not require disconnecting the needle from the syringe for inserting the guidewire into the blood vessel.

Disclosed herein are introducer components, assemblies, and methods that do not require disconnecting the needle from the syringe for inserting the guidewire into a blood vessel like typical introducer assemblies. Such introducer components, assemblies, and methods are advantageous in that they do not have the same risk of puncturing a backwall of the blood vessel or losing access to the blood vessel due to overhandling. In an example, an introducer assembly is disclosed including a fluidly connected syringe and needle. The needle includes a needle shaft and a needle hub over a proximal portion of the needle shaft. The needle hub includes a port and a valve disposed in the port. The port is in a side of the needle hub proximal of a proximal end of the needle shaft. The valve is configured to form a fluid-tight seal around an elongate medical device such as an access guidewire when passed through the port and into a needle-hub lumen of the needle hub. When the medical device is an access guidewire, the guidewire is disposed in the introducer assembly just proximal of a needle tip of the needle. In this way, the access guidewire is available to be immediately advanced into a blood-vessel lumen upon establishing a needle tract thereto with the needle. In another example, a method is disclosed for securing vascular access with the foregoing introducer assembly. Again, these and other features will become more apparent in view of the accompanying drawings and following description, which describe particular embodiments in greater detail.

### Introducer Assemblies

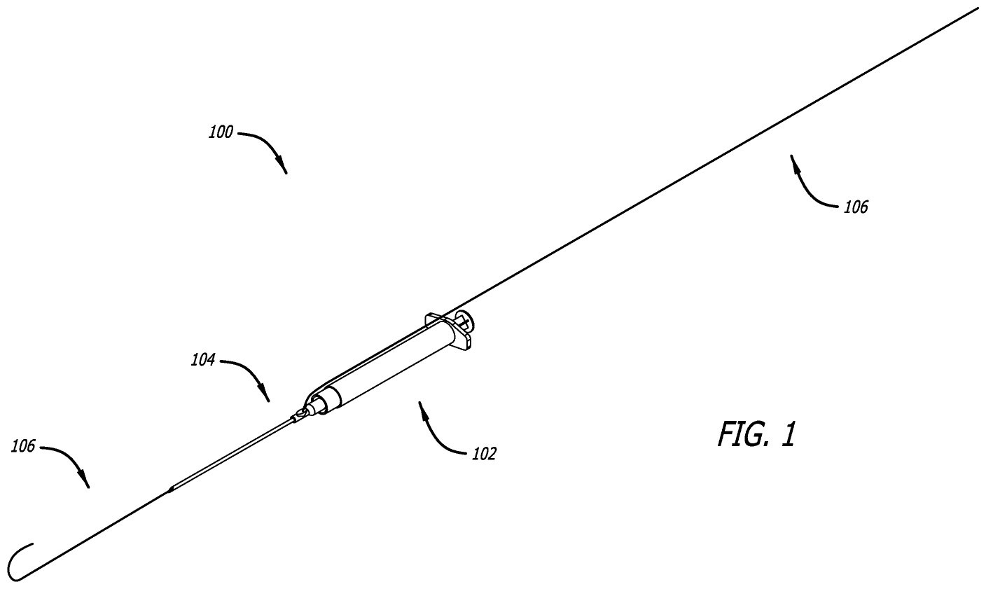

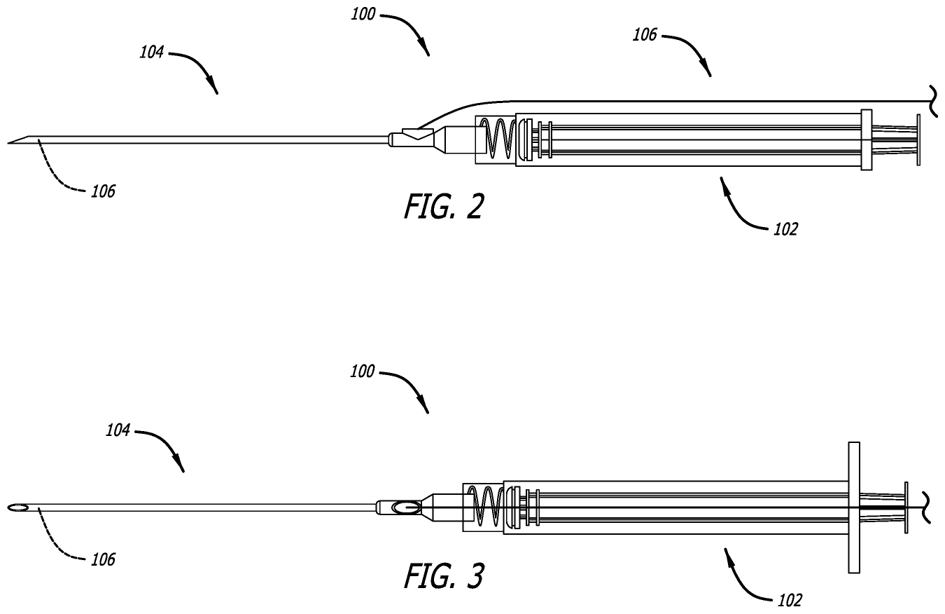

FIGS. 1-4 illustrate various view of an introducer assembly 100 in accordance with some embodiments.

As shown, the introducer assembly 100 includes a syringe 102 and a needle 104 fluidly connected to the syringe 102 in at least a ready-to-deploy state of the introducer assembly 100. In addition, the introducer assembly 100 can include an access guidewire 106 slidably disposed in the introducer assembly 100 in the ready-to-deploy state of the introducer assembly 100. Indeed, as set forth in more detail below, the access guidewire 106 passes through the port 144, through the needle-hub lumen 148, through the proximal end of the needle shaft 136, and into the needle-shaft lumen 142 of the needle shaft 136 such that the guidewire tip 154 is just proximal of the needle tip 138 in the ready-to-deploy state of the introducer assembly 100. In this way, the access guidewire 106 is available to be immediately advanced into a blood-vessel lumen upon establishing a needle tract thereto with the needle 104.

The syringe 102 includes a syringe hub 108, a barrel 110, and a plunger 112 disposed in the barrel 110 in at least the ready-to-deploy state of the introducer assembly 100.

The syringe hub 108 includes a syringe tip 114 extending from a distal portion (e.g., a distal end) of the barrel 110. In addition, the syringe hub 108 can include a threaded collar 116 extending from the distal portion (e.g., the distal end) of the barrel 110 around the syringe tip 114.

The syringe tip 114 is configured to insert into the needle-hub bore 152 of the needle hub 140 set forth below for fluidly connecting the syringe 102 to the needle 104. Indeed, the syringe tip 114 can have a Luer taper (e.g., a 6% taper) configured to insert into the needle-hub bore 152, which needle-hub bore 152 is complementarily configured as set forth below.

The threaded collar 116 includes internal threads 118 configured to screw together with the needle-hub flange of the needle hub 140 set forth below. When present, the threaded collar 116 of the syringe hub 108 advantageously provides a so-called Luer lock-style connection with the needle-hub flange of the needle hub 140 for added security against inadvertent disconnection over that provided by an otherwise Luer slip-style connection.

The barrel 110 includes a barrel wall 120, a barrel chamber 122 defined by the barrel wall 120, and a barrel flange 124, barrel collar, or the like outwardly extending from a proximal portion (e.g., a proximal end) of the barrel 110 or barrel wall 120 configured for actuating the syringe 102 together with the plunger flange 128, the plunger collar, or the like set forth below.

The barrel chamber 122 is configured to accept the plunger 112 when inserted therein. Indeed, the barrel chamber 122 extends from a distal end of the barrel 110, which is a closed end of the barrel 110 (excepting the syringe tip 114), to the proximal end of the barrel 110, which is an open end of the barrel 110 into which the plunger 112 can be inserted.

The plunger 112 includes a one-piece plunger shaft 126, a piston 127 fitted over a distal portion (e.g., a distal end) of the plunger shaft 126, and a plunger flange 128, a plunger collar, or the like outwardly extending from a proximal portion (e.g., a proximal end) of the plunger 112 configured for actuating the syringe 102 together with the barrel flange 124, barrel collar, or the like.

The plunger shaft 126 includes orthogonal struts 130 meeting along their longitudinal edges at a central axis of the plunger shaft 126.

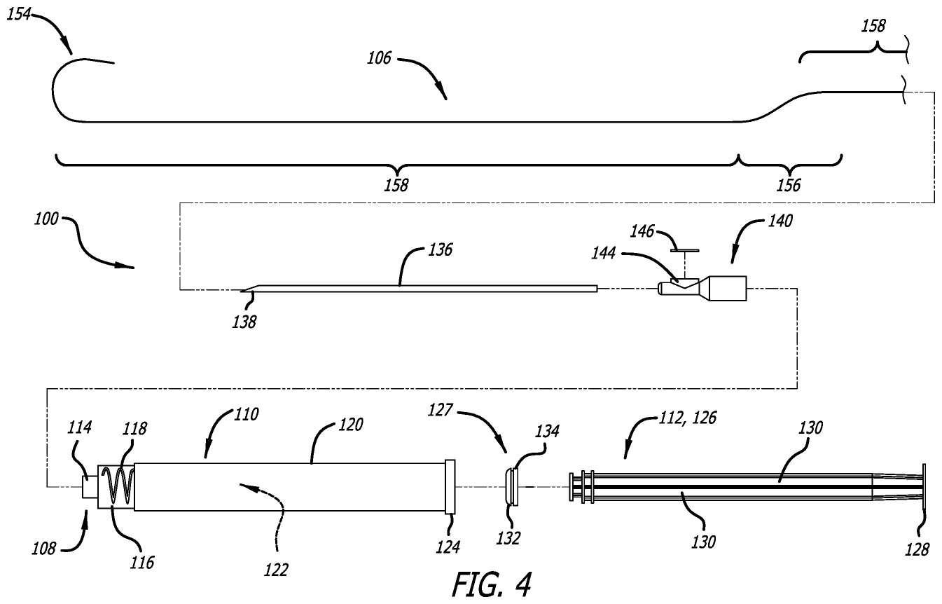

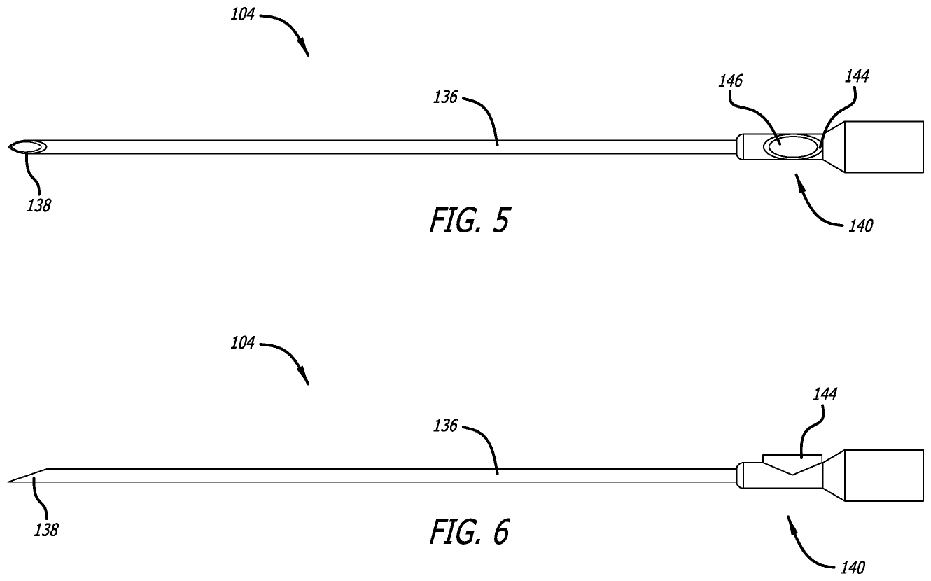

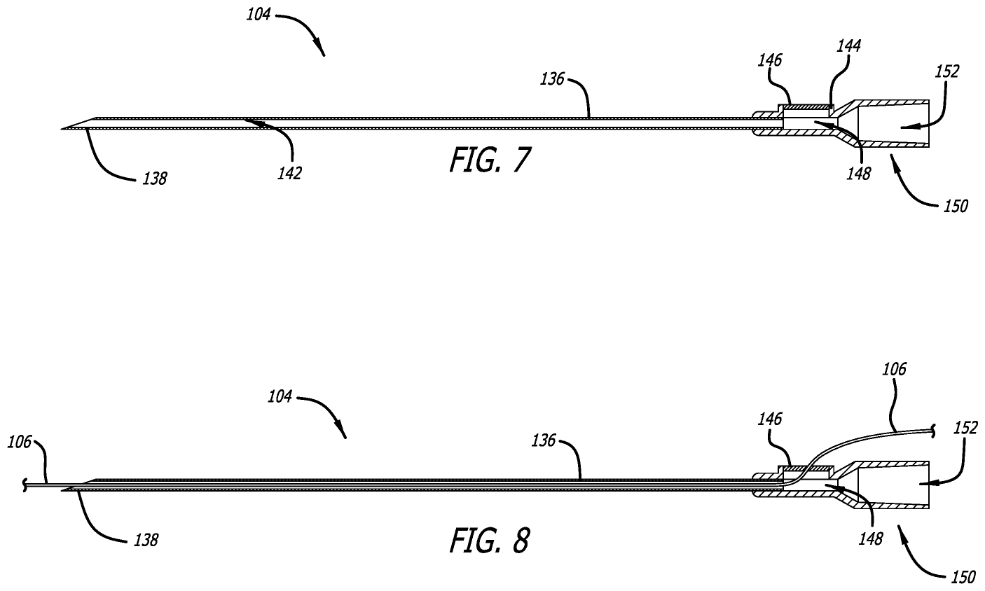

The piston 127, which can be an integral, elastomeric piston, includes one or more rings configured to respectively form one or more seals with the barrel wall 120. The one-or-more rings include at least a leading ring 132 configured to form a seal with the barrel wall 120. The one-or-more rings can also include a trailing ring 134 as shown in FIG. 4. Like the leading ring 132, the trailing ring 134 is configured to form a seal with the barrel wall 120. Indeed, the trailing ring 134, when present, provides a backup seal with the barrel wall 120. Together, the leading ring 132 and the trailing ring 134 ensure the seal (e.g., the seal provided by the leading ring 132, the trailing ring 134, or both the leading ring 132 and the trailing ring 134) between the piston 127 and the barrel wall 120 remains intact while the syringe 102 is actuated, thereby allowing the syringe 102 to consistently aspirate a liquid such as blood when the plunger 112 is withdrawn from the barrel 110.

The needle 104 includes a needle shaft 136, a needle tip 138 in a distal portion of the needle shaft 136, and a needle hub 140 over a proximal portion of the needle shaft 136.

The needle shaft 136 includes a needle-shaft lumen 142 extending from an opening in the needle tip 138 to a proximal end of the needle shaft 136.

The needle hub 140 includes a port 144, a valve 146 disposed in the port 144, and a needle-hub lumen 148 radially inward from the port 144. The port 144 is in a side of the needle hub 140 proximal of a proximal end of the needle shaft 136 such that the access guidewire 106 can be passed through the port 144, through a portion of the needle-hub lumen 148, and into the proximal end of the needle shaft 136. The valve 146, which can be a septum or split septum disposed in the port 144, is configured to form a fluid-tight seal around the access guidewire 106 when passed through the port 144 and into the needle-hub lumen 148 of the needle hub 140.

The needle hub 140 further includes a needle-hub connector 150, which, in turn, includes a needle-hub bore 152 proximal of the port 144 and an optional needle-hub flange about a proximal portion of the needle-hub connector 150.

The needle-hub bore 152 is configured to accept the syringe tip 114 therein for fluidly connecting the needle 104 to the syringe 102. Indeed, the needle-hub bore 152 can have a Luer taper (e.g., a 6% taper) configured to accept the syringe tip 114 therein, which syringe tip 114 is complementarily configured as set forth above.

While not shown, the needle-hub flange is configured to screw together with the internal threads 118 of the threaded collar 116 of the syringe hub 108. When present, the needle-hub flange advantageously provides a so-called Luer lock-style connection with the internal threads 118 of the threaded collar 116 of the syringe hub 108 for added security against inadvertent disconnection over that provided by an otherwise Luer slip-style connection.

Again, the introducer assembly 100 can include the access guidewire 106 slidably disposed in the introducer assembly 100 in the ready-to-deploy state of the introducer assembly 100. Indeed, the access guidewire 106 passes through the port 144, through the needle-hub lumen 148, through the proximal end of the needle shaft 136, and into the needle-shaft lumen 142 of the needle shaft 136 such that the guidewire tip 154 is just proximal of the needle tip 138 in the ready-to-deploy state of the introducer assembly 100. In this way, the access guidewire 106 is available to be immediately advanced into a blood-vessel lumen upon establishing a needle tract thereto with the needle 104. Notably, the access guidewire 106 includes a proximal portion proximally extending from the port 144 in the ready-to-deploy state of the introducer assembly 100. While not shown, the proximal portion of the access guidewire 106 can be disposed in a sterile barrier such as an elongate bag or even a guidewire-dispensing device, the sterile barrier or guidewire-dispensing device configured to maintain sterility of the access guidewire 106.

The access guidewire 106 can include a guidewire tip 154 in the form of a ‘J’-shaped guidewire tip configured to prevent puncturing a back wall of a blood vessel. Such a guidewire tip assumes a straightened state in the ready-to-deploy state of the introducer assembly 100 and a curved state when the guidewire tip 154 is advanced beyond the needle tip 138 in a deployed state of the introducer assembly 100.

The access guidewire 106 can further include a bare-wire portion 156 and a wound-wire portion 158 distal of the bare-wire portion 156, proximal of the bare-wire portion 156, or both. The bare-wire portion 156 distally extends through the valve 146 in at least the ready-to-deploy state of the introducer assembly 100 for forming the fluid-tight seal. Indeed, to maintain the fluid-tight seal even when a distal portion of the access guidewire 106 is advanced into a blood-vessel lumen, the bare-wire portion 156 can further distally extend through the valve 146 in one-or-more deployed states of the introducer assembly 100 as well. Notably, the access guidewire 106 need not have the bare-wire portion 156 and the wound-wire portion 158. At least the foregoing bare-wire portion 156 can instead be a flat-wound or ground-wound portion of the access guidewire 106, wherein the flat-wound portion includes windings of a tape instead of a round wire, and wherein the ground-wound portion includes windings of a round wire ground down to flatten the windings.

### Methods

Methods include at least a method for securing vascular access. Such a method includes one or more steps selected from an introducer assembly-obtaining step, an introducer assembly-adjusting step, a needle tract-establishing step, a plunger-withdrawing step, a blood-aspirating step, an access guidewire-advancing step, a syringe-and-needle disconnecting step, an air-bleeding step, and a needle-withdrawing step.

The introducer assembly-obtaining step includes obtaining the introducer assembly 100. As set forth above, the introducer assembly 100 includes the syringe 102, the needle 104 fluidly coupled to the syringe 102, and the access guidewire 106 slidably disposed in the introducer assembly 100. The needle 104 includes the needle hub 140 having the port 144 in the side of the needle hub 140. Again, the port 144 is proximal of the proximal end of the needle shaft 136 disposed in the needle hub 140. In at least the ready-to-deploy state of the introducer assembly 100, the access guidewire 106 passes through the valve 146 disposed in the port 144, through the needle-hub lumen 148 of the needle hub 140, through the proximal end of the needle shaft 136, and into the needle-shaft lumen 142 of the needle shaft 136.

The introducer assembly-adjusting step includes adjusting the introducer assembly 100 such that the introducer assembly 100 is in the ready-to-deploy state thereof if not already upon performing the introducer assembly-obtaining step. Again, in the ready-to-deploy state of the introducer assembly 100, the guidewire tip 154 in the distal portion of the access guidewire 106 is just proximal of the needle tip 138 in the distal portion of the needle shaft 136 for performing the access guidewire-advancing step immediately upon the establishing of the needle tract in the needle tract-establishing step.

The needle tract-establishing step includes establishing a needle tract from an area of skin to a blood-vessel lumen of a patient with the needle 104.

The plunger-withdrawing step includes withdrawing the plunger 112 from the barrel 110 of the syringe 102 to create a slight vacuum before reaching the blood-vessel lumen in the needle tract-establishing step. The slight vacuum ensures blood flashes back into at least the syringe tip 114 to confirm the establishing of the needle tract in the needle tract-establishing step.

The blood-aspirating step includes aspirating blood with the syringe 102 to confirm the establishing of the needle tract in the needle tract-establishing step. The valve 146 disposed in the port 144 is configured to form a fluid-tight seal around the bare-wire portion 156 of the access guidewire 106 for maintaining a vacuum during the blood-aspirating step.

The access guidewire-advancing step includes advancing the access guidewire 106 into the blood-vessel lumen for the securing of the vascular access. The access guidewire-advancing step allows the guidewire tip 154 of the access guidewire 106 to transition from the straightened state in the access-guidewire passageway to the curved state in the blood-vessel lumen.

The syringe-and-needle disconnecting step includes disconnecting the needle 104 from the syringe 102 before the needle-withdrawing step. However, the syringe-and-needle disconnecting step need not be performed if the air-bleeding step is performed.

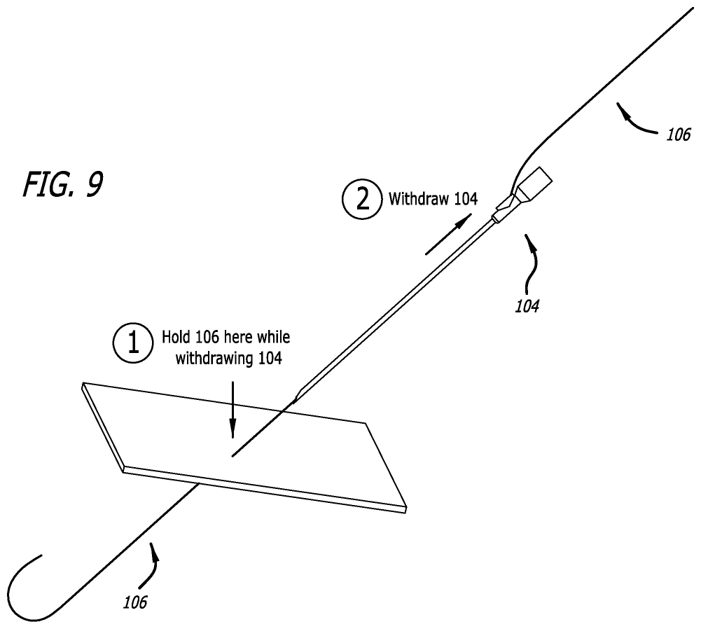

FIG. 9 illustrates the needle-withdrawing step in accordance with some embodiments.

The needle-withdrawing step includes withdrawing the needle 104 from the patient leaving the access guidewire 106 in the blood-vessel lumen. As shown, the needle-withdrawing step includes holding the guidewire in place at or near the area of skin including the needle tract while withdrawing the needle 104 over the proximal portion of the access guidewire 106.

The air-bleeding step includes bleeding air into the port 144 during the needle-withdrawing step by pushing the access guidewire 106 to a side of the port 144 against the valve 146; however, in some embodiments, the needle hub 140 includes a push-button bleed valve on another side of the needle hub 140 for the air-bleeding step. The bleeding of air into the port 144 obviates the disconnecting step of disconnecting the needle 104 from the syringe 102. That said, both the syringe-and-needle disconnecting step and the air-bleeding step can be performed in some embodiments.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An introducer assembly, comprising:
a syringe; and
a needle fluidly connected to the syringe, the needle including:
a needle shaft; and
a needle hub over a proximal portion of the needle shaft, the needle hub including:
a port in a side of the needle hub proximal of a proximal end of the needle shaft; and
a valve disposed in the port configured to form a fluid-tight seal around an elongate medical device when passed through the port and into a needle-hub lumen of the needle hub.

2. The introducer assembly of claim 1, further comprising an access guidewire slidably disposed in the introducer assembly in a ready-to-deploy state of the introducer assembly, the access guidewire passing through the port, through the needle-hub lumen, through the proximal end of the needle shaft, and into a needle-shaft lumen of the needle shaft such that a guidewire tip in a distal portion of the access guidewire is disposed just proximal of a needle tip in a distal portion of the needle shaft.

3. The introducer assembly of claim 2, wherein the guidewire tip is a T-shaped guidewire tip that assumes a straightened state in the ready-to-deploy state of the introducer assembly and a curved state when the guidewire tip is advanced beyond the needle tip in a deployed state of the introducer assembly.

4. The introducer assembly of claim 2, wherein the access guidewire includes a bare-wire portion and a wound-wire portion proximal of the bare-wire portion, the bare-wire portion distally extending through the valve in at least the ready-to-deploy state of the introducer assembly for forming the fluid-tight seal.

5. The introducer assembly of claim 2, wherein the access guidewire includes a proximal portion proximally extending from the port in the ready-to-deploy state of the introducer assembly, the proximal portion of the access guidewire disposed in a sterile barrier configured to maintain sterility of the access guidewire.

6. The introducer assembly of claim 1, wherein the valve includes a split septum compressed in the port.

7. The introducer assembly of claim 1, wherein the needle hub further includes a needle-hub connector including a needle-hub bore proximal of the port, a syringe tip of the syringe disposed in the needle-hub bore, thereby fluidly connecting the needle to the syringe.

8. An introducer needle, comprising:
a needle shaft; and
a needle hub over a proximal portion of the needle shaft, the needle hub including:
a port in a side of the needle hub proximal of a proximal end of the needle shaft; and
a valve disposed in the port configured to form a fluid-tight seal around an elongate medical device when passed through the port and into a needle-hub lumen of the needle hub.

9. The introducer needle of claim 8, wherein the valve includes a split septum compressed in the port.

10. The introducer needle of claim 8, wherein the needle hub further includes a needle-hub connector including a needle-hub bore proximal of the port configured to accept a syringe tip inserted therein for fluidly connecting the needle to the syringe.

11. A method for securing vascular access, comprising:
obtaining an introducer assembly, the introducer assembly including:
a syringe;
a needle fluidly coupled to the syringe, the needle including needle hub having a port in a side of the needle hub proximal of a proximal end of a needle shaft disposed in the needle hub; and
an access guidewire slidably disposed in the introducer assembly, the access guidewire passing through a valve disposed in the port, through a needle-hub lumen of the needle hub, through the proximal end of the needle shaft, and into a needle-shaft lumen of the needle shaft;
establishing a needle tract from an area of skin to a blood-vessel lumen of a patient with the needle; and
advancing the access guidewire into the blood-vessel lumen for the securing of the vascular access.

12. The method of claim 11, further comprising adjusting the introducer assembly such that the introducer assembly is in a ready-to-deploy state thereof with a guidewire tip in a distal portion of the access guidewire disposed just proximal of a needle tip in a distal portion of the needle shaft for the advancing of the access guidewire into the blood-vessel lumen immediately upon the establishing of the needle tract.

13. The method of claim 12, wherein the advancing of the access guidewire into the blood-vessel lumen allows the guidewire tip of the access guidewire to transition from a straightened state in the access-guidewire passageway to a curved state in the blood-vessel lumen.

14. The method of claim 11, further comprising withdrawing a plunger from a barrel of the syringe to create a slight vacuum before reaching the blood-vessel lumen, the slight vacuum ensuring blood flashes back into at least a syringe tip to confirm the establishing of the needle tract.

15. The method of claim 11, further comprising aspirating blood with the syringe to confirm the establishing of the needle tract, the valve disposed in the port configured to form a fluid-tight seal around a bare-wire portion of the access guidewire for maintaining a vacuum during the aspirating of the blood with the syringe.

16. The method of claim 11, further comprising withdrawing the needle from the patient leaving the access guidewire in the blood-vessel lumen.

17. The method of claim 16, wherein the withdrawing of the needle includes holding the guidewire in place at or near the area of skin including the needle tract while withdrawing the needle over a proximal portion of the access guidewire.

18. The method of claim 16, further comprising disconnecting the needle from the syringe before withdrawing the needle from the patient.

19. The method of claim 16, further comprising bleeding air into the port while withdrawing the needle from the patient by pushing the access guidewire to a side of the port against the valve, the bleeding of air into the port obviating disconnecting the needle from the syringe.

20. The method of claim 19, wherein the valve includes a split septum compressed in the port.
